SEQUENCE LISTING (12 sequences)

SEQ ID NO: 1 - protein, 15 aa, Artificial Sequence
  source: /note=“Description of Artificial Sequence Synthetic peptide”
  SITE (1)..(15): /note=“This sequence may encompass 1-3 ′Gly Gly Gly Gly Ser′ repeating units”
GGGGSGGGGSGGGGS

SEQ ID NO: 2 - protein, 5 aa, Artificial Sequence
  source: /note=“Description of Artificial Sequence Synthetic peptide”
RSKLG

SEQ ID NO: 3 - protein, 727 aa, Clostridium phage phiCPV4
MDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDVEEPKTFHIVQNNNIVYGCNVYMDKFFEVENKDGSTTEIGLCLDFFDSYKIITCAESQFHNYVHDVDPMFYKMGEEYDYDTWRSPTHKQTTLELRYQYNDIYMLREVIEQFYIDGLCGGELPLTGMRTASSIAFNVLKKMTFGEEKTEEGYINYFELDKKTKFEFLRKRIEMESYTGGYTHANHKAVGKTINKIGCSLDINSSYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKPYVDHFTKMKVENKKLGNKPLTNQAKLILNGAYGKFGTKQNKEEKDLIMDKNGLLTFTGSVTEYEGKEFYRPYASFVTAYGRLQLWNAIIYAVGVENFLYCDTDSIYCNREVNSLIEDMNAIGETIDKTILGKWDVEHVFDKFKVLGQKKYMYHDCKEDKTDLKCCGLPSDARKIIIGQGFDEFYLGKNVEGKKQRKKVIGGCLLLDTLFTIKKIMF

SEQ ID NO: 4 - protein, 305 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 5 - protein, 10 aa, Artificial Sequence
  source: /note=“Description of Artificial Sequence Synthetic peptide”
AHIVMVDAYK

SEQ ID NO: 6 - protein, 9 aa, Artificial Sequence
  source: /note=“Description of Artificial Sequence Synthetic peptide”
GSSGGSSGG

SEQ ID NO: 7 - protein, 13 aa, Artificial Sequence
  source: /note=“Description of Artificial Sequence Synthetic peptide”
AHIVMVDAYKPTK

SEQ ID NO: 8 - protein, 8 aa, Artificial Sequence
  source: /note=“Description of Artificial Sequence Synthetic peptide”
KGHHHHHH

SEQ ID NO: 9 - protein, 30 aa, Artificial Sequence
  source: /note=“Description of Artificial Sequence Synthetic polypeptide”
GSSGGSSGGAHIVMVDAYKPTKKGHHHHHH

SEQ ID NO: 10 - DNA, 5466 bp, Artificial Sequence
  source: /note=“Description of Artificial Sequence Synthetic polynucleotide”
cagtcagtagagagagagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctctctctcaaaaacggaggaggaggacagtcagtagagagagagatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatctctctctcaaaaacggaggaggagga

SEQ ID NO: 11 - DNA, 36 bp, Artificial Sequence
  source: /note=“Description of Artificial Sequence Synthetic oligonucleotide”
atctctctctactgactgtcctcctcctccgttttt

SEQ ID NO: 12 - DNA, 10 bp, Artificial Sequence
  source: /note=“Description of Artificial Sequence Synthetic oligonucleotide”
gagagagatt

(19) United States
(12) Patent Application Publication
(10) Pub. No.: US 2021/0381041 A1
(43) Pub. Date: Dec. 9, 2021

(54) Enzymatic Enrichment of DNA-Pore-Polymerase Complexes

(71) Applicant: Roche Sequencing Solutions, Inc., Pleasanton, CA (US)

(72) Inventors: Kirti DHIMAN, Mt. View, CA (US); Helen FRANKLIN, San Jose, CA (US); Alexandra WANG, Santa Clara, CA (US)

(21) Appl. No.: 17/058,019

(22) PCT Filed: May 27, 2019
(86) PCT No.: PCT/EP2019/063673

§ 371 Date: Nov. 23, 2020

Related U.S. Application Data
(60) Provisional application No. 62/677,142, filed on May 28, 2018.

(51) Int. Cl. C12Q 1/6869

(52) U.S. Cl. CPC C12Q 1/6869

(57) ABSTRACT

The present invention provides a method for isolating Sequencing complexes, said method comprising forming a complex between a nanopore covalently linked to a polymerase and an oligonucleotide that is associated with a purification moiety, separating any unbound/uncomplexed nanopores and oligonucleotides from the complexes by use of a solid support capable of binding the purification moiety, and cleaving bound complexes from the solid support with an enzyme composition.

### TECHNICAL FIELD

Disclosed are methods for isolating polymerase complexes that are subsequently incorporated into membranes of biochips to enable nanopore sequencing of polynucleotides. Also disclosed is a nucleic acid adaptor for isolating active polymerase complexes, polymerase complexes comprising the nucleic acid adaptor, and methods for isolating active polymerase complexes using the nucleic acid adaptor.

### BACKGROUND

Nanopores have recently emerged as a label-free platform for interrogating sequence and structure in nucleic acids. Data are typically reported as a time series of ionic current changes correlated to the DNA sequence as it is determined by applying an electric field across a single pore controlled by a voltage-clamped amplifier. Hundreds to thousands of molecules can be examined at high bandwidth and spatial resolution.

Obstacles to the success of nanopores as a reliable DNA analysis tool are obtaining a sufficient number of functional sequencing complexes to allow the use of the majority of the wells on a biochip and sequencing long polymers such as kilobase length or more (single-stranded genomic DNA or RNA) or small molecules (e.g., nucleosides) require amplification or labeling.

Thus, there is a need for sequencing complexes that allow for improved sequencing yield (e.g., improved numbers of functional sequencing complexes on a biochip), as well as the sequencing or identification without amplification or labeling of a template polynucleotide.

### SUMMARY

The present disclosure provides a method for isolating Sequencing complexes, said method comprising forming a complex between a nanopore covalently linked to a polymerase and an oligonucleotide that is associated with a purification moiety, separating any unbound/uncomplexed nanopores and oligonucleotides from the complexes by use of a solid support capable of binding the purification moiety, and cleaving bound complexes from the solid support with an enzyme composition.

In some embodiments, the method comprises (a) annealing an Enrichment Primer to a sample DNA to form an Annealed Template Oligonucleotide; (b) purifying the Annealed Template Oligonucleotide; (c) combining a Conjugate with the Annealed Template Oligonucleotide to form an Eintopf; (d) combining the Eintopf with a solid support capable of binding to the Purification Moiety to produce an Enriched Eintopf; and (e) cleaving the linker with an enzyme composition to release the Purification Moiety thereby releasing the Sequencing Complex to provide an Enriched Sequencing Complex solution.

In some embodiments, the method comprises (a) combining a Conjugate with an Annealed Template Oligonucleotide comprising a Purification Moiety to form an Eintopf and allowing the Conjugate to bind the Annealed Template Oligonucleotide to form a Sequencing Complex; (b) combining the Eintopf with a solid support capable of binding to the Purification Moiety of the Annealed Template Oligonucleotide, (c) separating the unbound Complex Components from the bound Sequencing Complexes; (d) cleaving the linker with an enzyme composition to release the Purification Moiety, wherein the purification moiety remains associated with the solid support, thereby releasing the Sequencing Complex, and (e) separating the solid support from the Sequencing Complex to provide an Enriched Sequencing Complex solution.

In all embodiments, the Enrichment Primer comprises an oligonucleotide that is complementary to a portion of an adaptor, an enzymatically cleavable linker and a Purification Moiety. In all embodiments, the linker comprises an abasic site or at least one uracil residue.

In all embodiments, the sample DNA is either linear, circular or self-priming. In some embodiments, the sample DNA has been ligated to at least one adaptor. In some embodiments, an adaptor has been ligated to each end of the sample DNA. In some embodiments, the adaptors are dumbbell adaptors. In all embodiments, the adaptor comprises a primer recognition sequence capable of binding to the Enrichment Primer.

In some embodiments, purifying the Annealed Template Oligonucleotide comprises binding to a solid support that selectively binds double stranded DNA. In some embodiments, the Annealed Template Oligonucleotide is not purified before being complexed with a Conjugate. In all embodiments, the double-stranded DNA is greater than 100 base pairs in length, greater than 500 bp in length or greater than 1000 bp in length. In some embodiments, the double-stranded DNA is a concatemer of multiple DNA fragments. In all embodiments, the sample DNA comprises a barcode comprising a sample identifier and/or a patient identifier. In some embodiments, the solid support comprises beads. In some embodiments, the beads are paramagnetic beads. In some embodiments, the beads comprise carboxyl moieties.

In some embodiments, the solid support capable of binding to the Purification Moiety is a bead. In some embodiments, the beads comprise streptavidin. In some embodiments, the beads are paramagnetic beads.

In all embodiments, the concentration of the Sequencing Complex in the solution is greater than 70%, greater than 75%, greater than 80%.

In all embodiments, the enzyme composition comprises an Endonuclease VIII, an Endonuclease III, a lyase, a glycolyase, or combinations thereof.

In an embodiment, a method for preparing a biochip, said method comprising (a) isolating a Sequencing Complex; (b) flowing the Sequencing Complex over a lipid bilayer of said biochip; and (c) applying a voltage to said chip sufficient to insert a Sequencing Complex in the lipid bilayer. In some embodiments, the biochip has a density of said nanopore sequencing complexes of at least 500,000 nanopore sequencing complexes 1 mm². In some embodiments, at least 70% of the Sequencing Complexes are functional nanopore-polymerase complexes.

### BRIEF DESCRIPTION OF THE FIGURES

FIG. 1 is a cartoon of the various components described in the present disclosure and used in the present methods.

FIG. 2 is a cartoon of the method described herein.

FIG. 3 depicts the 5466 bp sequence of the pUC19 dumbbell DNA template used in the nanopore detection methods.

FIG. 4 depicts a linear adapter (top) and a HEG adapter (bottom). Figure discloses SEQ ID NOS 11, 12, 11, and 12, respectively, in order of appearance.

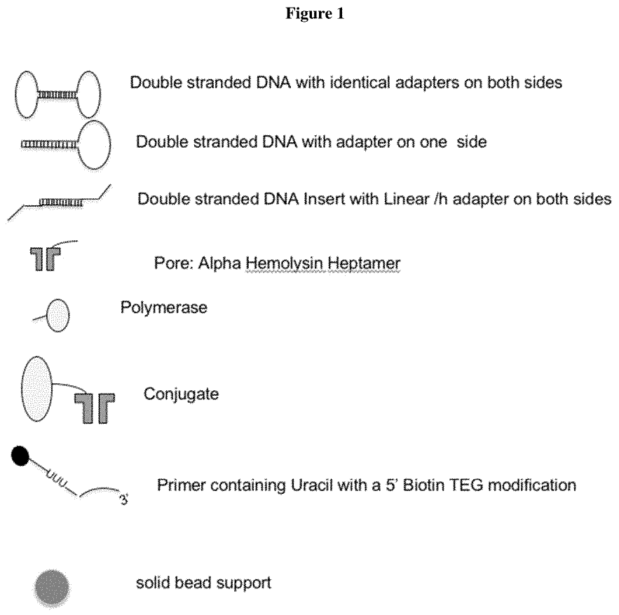

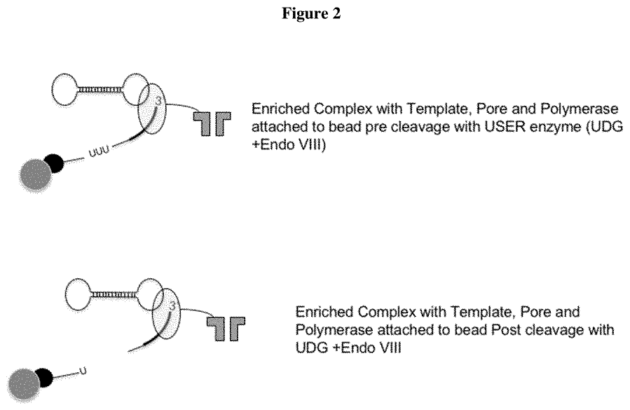

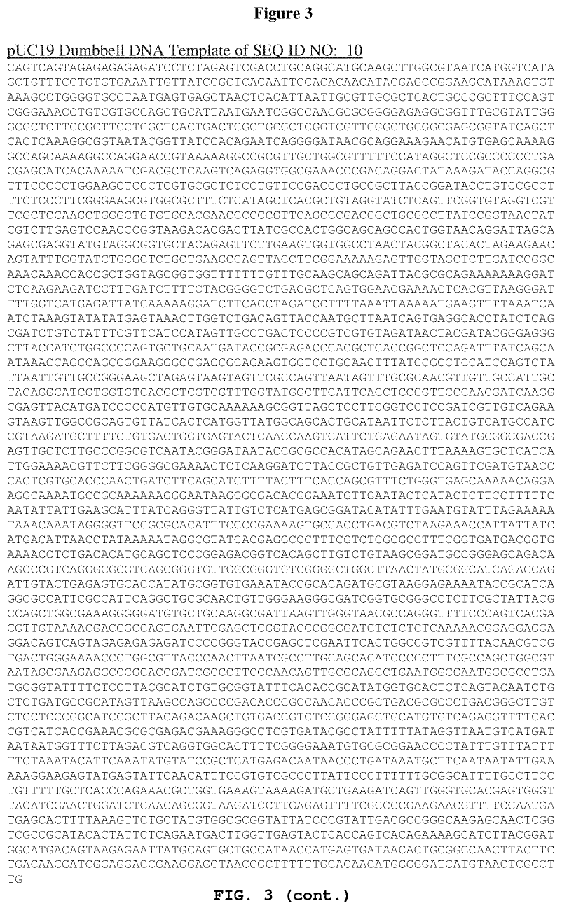

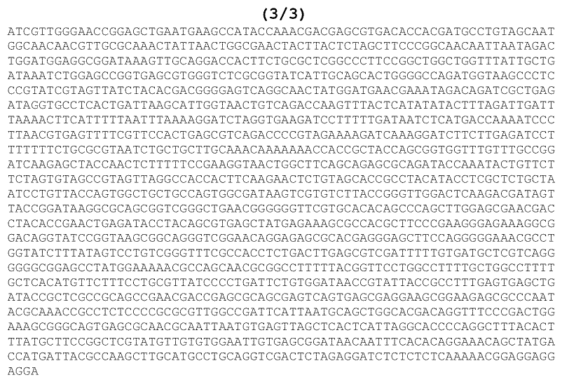

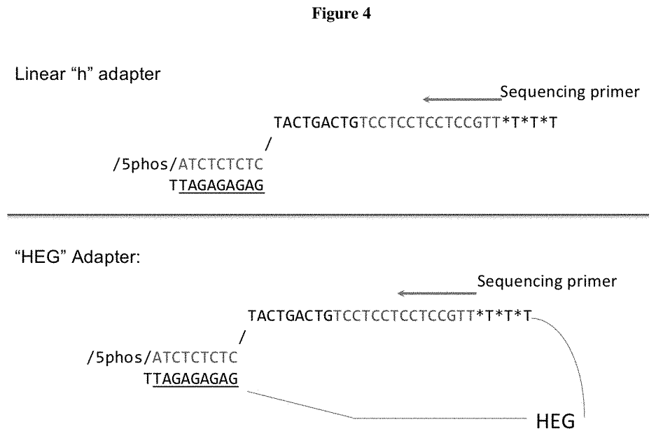

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50” includes “2 to 25”, “5 to 20”, “25 to 50”, “1 to 10”, etc.

It is to be understood that both the foregoing general description, including the drawings, and the following detailed description are exemplary and explanatory only and are not restrictive of this disclosure.

### Definitions

“Nucleic acid,” as used herein, refers to a molecule of one or more nucleic acid subunits which comprise one of the nucleobases, adenine (A), cytosine (C), guanine (G), thymine (T), and uracil (U), or variants thereof. Nucleic acid can refer to a polymer of nucleotides (e.g., dAMP, dCMP, dGMP, dTMP), also referred to as a polynucleotide or oligonucleotide, and includes DNA, RNA, in both single and double-stranded form, and hybrids thereof.

“Nucleotide,” as used herein refers to a nucleoside-5′-oligophosphate compound, or structural analog of a nucleoside-5′-oligophosphate, which is capable of acting as a substrate or inhibitor of a nucleic acid polymerase. Exemplary nucleotides include, but are not limited to, nucleoside-5′-triphosphates (e.g., dATP, dCTP, dGTP, dTTP, and dUTP); nucleosides (e.g., dA, dC, dG, dT, and dU) with 5′-oligophosphate chains of 4 or more phosphates in length (e.g., 5′-tetraphosphosphate, 5′-pentaphosphosphate, 5′-hexaphosphosphate, 5′-heptaphosphosphate, 5′-octaphosphosphate); and structural analogs of nucleoside-5′-triphosphates that can have a modified nucleobase moiety (e.g., a substituted purine or pyrimidine nucleobase), a modified sugar moiety (e.g., an O-alkylated sugar), and/or a modified oligophosphate moiety (e.g., an oligophosphate comprising a thio-phosphate, a methylene, and/or other bridges between phosphates).

“Nucleoside,” as used herein, refers to a molecular moiety that comprises a naturally occurring or non-naturally occurring nucleobase attached to a sugar moiety (e.g., ribose or deoxyribose).

“Polymerase,” as used herein, refers to any natural or non-naturally occurring enzyme or other catalyst that is capable of catalyzing a polymerization reaction, such as the polymerization of nucleotide monomers to form a nucleic acid polymer. Exemplary polymerases that may be used in the compositions and methods of the present disclosure include the nucleic acid polymerases such as DNA polymerase (e.g., enzyme of class EC 2.7.7.7), RNA polymerase (e.g., enzyme of class EC 2.7.7.6 or EC 2.7.7.48), reverse transcriptase (e.g., enzyme of class EC 2.7.7.49), and DNA ligase (e.g., enzyme of class EC 6.5.1.1).

“Moiety,” as used herein, refers to part of a molecule.

“Linker,” as used herein, refers to any molecular moiety that provides a bonding attachment with some space between two or more molecules, molecular groups, and/or molecular moieties.

“Tag,” as used herein, refers to a moiety or part of a molecule that enables or enhances the ability to detect and/or identify, either directly or indirectly, a molecule or molecular complex, which is coupled to the tag. For example, the tag can provide a detectable property or characteristic, such as steric bulk or volume, electrostatic charge, electrochemical potential, optical and/or spectroscopic signature.

“Nanopore,” as used herein, refers to a pore, channel, or passage formed or otherwise provided in a membrane or other barrier material that has a characteristic width or diameter of about 1 angstrom to about 10,000 angstroms. A nanopore can be made of a naturally-occurring pore-forming protein, such as α-hemolysin from S. aureus, or a mutant or variant of a wild-type pore-forming protein, either non-naturally occurring (i.e., engineered) such as α-HL-C46, or naturally occurring. A membrane may be an organic membrane, such as a lipid bilayer, or a synthetic membrane made of a non-naturally occurring polymeric material. The nanopore may be disposed adjacent or in proximity to a sensor, a sensing circuit, or an electrode coupled to a sensing circuit, such as, for example, a complementary metal-oxide semiconductor (CMOS) or field effect transistor (FET) circuit.

“Nanopore-detectable tag” as used herein refers to a tag that can enter into, become positioned in, be captured by, translocate through, and/or traverse a nanopore and thereby result in a detectable change in current through the nanopore. Exemplary nanopore-detectable tags include, but are not limited to, natural or synthetic polymers, such as polyethylene glycol, oligonucleotides, polypeptides, carbohydrates, peptide nucleic acid polymers, locked nucleic acid polymers, any of which may be optionally modified with or linked to chemical groups, such as dye moieties, or fluorophores, that can result in detectable nanopore current changes.

“Ion flow,” as used herein, refers to the movement of ions, typically in a solution, due to an electromotive force, such as the potential between an anode and a cathode. Ion flow typically can be measured as current or the decay of an electrostatic potential.

“Ion flow altering,” as used herein in the context of nanopore detection, refers to the characteristic of resulting in a decrease or an increase in ion flow through a nanopore relative to the ion flow through the nanopore in its “open channel” (O.C.) state.

“Open channel current,” “O.C. current,” or “Background current” as used herein refers to the current level measured across a nanopore when a potential is applied and the nanopore is open (e.g., no tag is present in the nanopore).

“Tag current” as used herein refers to the current level measured across a nanopore when a potential is applied and a tag is present the nanopore. For example, depending on a tag's specific characteristics (e.g., overall charge, structure, etc.), the presence of the tag in a nanopore can decrease ion flow through the nanopore and thereby result in a decrease in measured tag current level.

“Complex Components” as used herein refers to unbound components required to form a Sequencing Complex. The components include a polynucleotide template associated with a Purification Moiety, and a polymerase-nanopore complex. The polynucleotide template may be annealed to an oligonucleotide primer when the polynucleotide template or adaptor is not self-priming, wherein the oligonucleotide primer comprises a purification moiety. The oligonucleotide primer, or self-priming template or self-priming adaptor further comprise a purification moiety.

“Eintopf” as used herein refers to a complex comprising the elements or consisting of the elements for a Sequencing Complex, i.e., an Annealed Template (e.g., template-primer hybrid), and a Conjugate. Once the Annealed Template and the Conjugate associate in the solution then the Sequencing Complex may be isolated therefrom to provide an Enriched Sequencing Complex.

“Polymerase complex” as used herein refers to a complex formed by the association of a polymerase enzyme and a polynucleotide template substrate. Polynucleotide templates that are not self-priming require oligonucleotide primers to initiate strand extension. Accordingly, absent a self-priming polynucleotide, a polymerase complex can further include an oligonucleotide primer, which may comprise a purification moiety.

“Enriched Sequencing Complex” as used herein refers to a solution comprising a Sequencing Complex that has been enriched from a solution comprising an Eintopf such that complex components that have not become associated to form a Sequencing Complex have been removed to result in a solution that is at least 70%, 75%, 80%, or 85% by weight Sequencing Complex.

“Conjugate” as used herein refers to a nanopore covalently linked to a polymerase.

“Capture complex” as used herein refers to a complex formed by the association of a polymerase enzyme, a polynucleotide template, and a capture oligonucleotide.

“Capture oligonucleotide” or “Enrichment Primer” are used interchangeably and as used herein refer to an oligonucleotide that comprises a purification moiety that serves to immobilize a Sequencing Complex with which it is associated to a solid support. Preferably, the purification moiety can be biotin or modified biotin, which binds to a purification moiety-binding partner, e.g., streptavidin or modified streptavidin, on the solid support. If the template oligonucleotide is self-priming, the purification moiety is incorporated into the self-priming template (as provided for below), and thus when associated with a Conjugate is a Capture oligonucleotide.

“Polynucleotide template” and “polynucleotide substrate template” as used herein refer to a polynucleotide molecule from which a complementary nucleic acid strand is synthesized by a polynucleotide polymerase, e.g., DNA polymerase. The polynucleotide template can be linear, hairpin, or continuous. Continuous templates can be circular or dumbbell. Hairpin templates can be self-priming templates or comprise a universal priming sequence.

“Primed template” and “Annealed Template” as used refers to a template oligonucleotide that is associated with a purification moiety. Thus, if the template oligonucleotide is self-priming, the purification moiety is incorporated into the self-priming template. In addition, if adaptors have been ligated to the template oligonucleotide and the adaptors are self-priming, then the purification moiety is incorporated into the self-priming adaptor. Finally, if the template oligonucleotide has been annealed to a primer, the primer comprises a purification moiety.

“Purification Moiety” as used herein refers to a moiety that aids in the purification of a Sequencing Complex.

“Sequencing Complex” as used herein refers to a pore covalently linked to a DNA polymerase which is hound to a primed template DNA, e.g., an Annealed Template.

“Enriched” as used herein refers to a molecule that is present in a sample at a concentration of at least 75% by weight, or at least 80% by weight of the sample in which it is contained.

“Biotinylated” as used herein refers to a modified molecule, e.g., nucleic acid molecules (including single or double stranded DNA, RNA, DNA/RNA chimeric molecules, nucleic acid analogs and any molecule which contains or incorporates a nucleotide sequence, e.g., a peptide nucleic acid (PNA) or any modification thereof), proteins (including glycoproteins, enzymes, peptide library or display products and antibodies or derivatives thereof), peptides, carbohydrates or polysaccharides, lipids, etc., wherein the molecules are covalently linked to a biotin or biotin analogue. Many biotinylated ligands are commercially available or can be prepared by standard methods. Processes for coupling a biomolecule, e.g., a nucleic acid molecule or a protein molecule, to biotin are well known in the art (Bayer and Wilchek, “Avidin-Biotin Technology: Preparation of Biotinylated Probes”, Methods in Molec. Biology 10, 137-148. 1992).

“Binding partner” as used refers to any biological or other organic molecule capable of specific or non-specific binding or interaction with another biological molecule, which binding or interaction may be referred to as “ligand” binding or interaction and is exemplified by, but not limited to, antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector or repressor/inducer bindings or interactions. The term “binding partner” herein refers to the partners of an affinity complex, e.g., biotin-biotin-binding partner, used in the isolation methods described herein.

“Biotin-binding” compound as used herein is intended to encompass any compound which is capable of tightly but non-covalently binding to biotin or any biotin compound. Preferred biotin-binding compounds include modified streptavidin and avidin, as well as derivatives and analogues thereof, e.g., nitro-streptavidin.

“Avidin” as used herein refers to the native egg-white glycoprotein avidin as well as derivatives or equivalents thereof, such as deglycosylated or recombinant forms of avidin, for example, N-acyl avidins, e.g., N-acetyl, N-phthalyl and N-succinyl avidin, and the commercial products ExtrAvidin, Neutralite Avidin and CaptAvidin

“Streptavidin” as used herein refers to bacterial streptavidins produced by selected strains of Streptomyces, e.g., Streptomyces avidinii, as well as derivatives or equivalents thereof such as recombinant and truncated streptavidin, such as, for example, “core” streptavidin.

### Template Polynucleotides

The methods and compositions provided herein are applicable to various different kinds of nucleic acid templates, nascent strands, and double-stranded products, including single-stranded DNA; double-stranded DNA; single-stranded RNA; double-stranded RNA; DNA-RNA hybrids; nucleic acids comprising modified, missing, unnatural, synthetic, and/or rare nucleosides; and derivatives, mimetics, and/or combinations thereof.

The template nucleic acids of the invention can comprise any suitable polynucleotide, including double-stranded DNA, single-stranded DNA, single-stranded DNA hairpins, DNA/RNA hybrids, RNAs with a recognition site for binding of the polymerizing agent, and RNA hairpins. Further, target polynucleotides may be a specific portion of a genome of a cell, such as an intron, regulatory region, allele, variant or mutation; the whole genome; or any portion thereof. In other embodiments, the target polynucleotides may be, or be derived from mRNA, tRNA, rRNA, ribozymes, antisense RNA or RNAi.

The template nucleic acids (e.g., polynucleotides) can include unnatural nucleic acids such as PNAs, modified oligonucleotides (e.g., oligonucleotides comprising nucleotides that are not typical to biological RNA or DNA, such as 240-O-methylated oligonucleotides modified phosphate backbones and the like. A nucleic acid can be, e.g., single-stranded or double-stranded.

The nucleic acids used to produce the template nucleic acids in the methods herein (i.e., the target nucleic acids) may be essentially any type of nucleic acid amendable to the methods presented herein. In some cases, the target nucleic acid itself comprises the fragments that can be used directly as the template nucleic acid. Typically, the target nucleic acid will be fragmented and further treated (e.g., ligated with adaptors and or circularized) for use as templates. For example, a target nucleic acid may be DNA (e.g., genomic DNA, mtDNA, etc.), RNA (e.g., mRNA, siRNA, etc.), cDNA, peptide nucleic acid (PNA), amplified nucleic acid (e.g., via PCR, LCR, or whole genome amplification (WGA)), nucleic acid subjected to fragmentation and/or ligation modifications, whole genomic DNA or RNA, or derivatives thereof (e.g., chemically modified, labeled, recoded, protein-bound or otherwise altered).

The template nucleic acid may be linear, circular (including templates for circular redundant sequencing (CRS)), single- or double-stranded, and/or double-stranded with single-stranded regions (e.g., stem- and loop-structures). The template nucleic acid may be purified or isolated from an environmental sample (e.g., ocean water, ice core, soil sample, etc.), a cultured sample (e.g., a primary cell culture or cell line), samples infected with a pathogen (e.g., a virus or bacterium), a tissue or biopsy sample, a forensic sample, a blood sample, or another sample from an organism, e.g., animal, plant, bacteria, fungus, virus, etc. Such samples may contain a variety of other components, such as proteins, lipids, and non-target nucleic acids. In certain embodiments, the template nucleic acid is a complete genomic sample from an organism. In other embodiments, the template nucleic acid is total RNA extracted from a biological sample or a cDNA library. In some embodiments, the template DNA is a cell-free DNA (cfDNA) sample obtained from a blood or plasma sample. In some embodiments, the blood sample comprises fetal DNA.

In some embodiments, the template DNA is ligated to adapters. The adapters may be linear adapters, dumbbell adapters, hexaethylene glycol (HEG) adapters, etc. In some embodiments, the adapters comprise a sequence capable of annealing with a capture oligonucleotide. The HEG adapter comprises an 18 atom spacer that blocks polymerase activity. Therefore, the polymerase doesn't read both strands as it does for traditional dumbbell adaptors. Dumbbell adapters are well known in the art and are described elsewhere; see for example U.S. Pat. No. 8,153,375 (Pacific Biosciences).

### Polymerases

The polymerase of the Sequencing Complex can be a wild-type or a variant polymerase that retains polymerase activity under conditions used for sequencing. Examples of polymerases that find use in the compositions and methods described herein include phi29, po16, and variants thereof such as exo-nuclease deficient polymerases, and/or variant polymerases with altered kinetic characteristics. In some embodiments, the polymerase is a Pol6 polymerase that has an amino acid sequence that is at least 70% identical to SEQ ID NO: 3.

(Wild-type Po16 (DNA polymerase [Clostridium
phage phiCPV4]; GenBank: AFH27113.1)
SEQ ID NO: 3

| | |
|---|---|
| 1 | mdkhtqyvke hsfnydeykk anfdkiecli fdtesctnye |
| | ndntgarvyg wglgvtrnhn |
| 061 | miygqnlnqf wevcgnifnd wyhdnkhtik itktkkgfpk |
| | rkyikfpiav hnlgwdvefl |
| 121 | kyslvengfn ydkgllktvf skgapyqtvt dveepktfhi |
| | vqnnnivygc nvymdkffev |
| 181 | enkdgsttei glcldffdsy kiitcaesqf hnyvhdvdpm |
| | fykmgeeydy dtwrspthkq |
| 241 | ttlelryqyn diymlrevie qfyidglcgg elpltgmrta |
| | ssiafnvlkk mtfgeektee |
| 301 | gyinyfeldk ktkfeflrkr iemesytggy thanhkavgk |
| | tinkigcsld inssypsqma |
| 361 | ykvfpygkpv rktwgrkpkt eknevyliev gfdfvepkhe |
| | eyaldifkig avnskalspi |
| 421 | tgaysgqeyf ctnikdgkai pvykelkdtk lttnynvvlt |
| | sveyefwikh fnfgvfkkde |
| 481 | ydcfevdnle ftglkigsil yykaekgkfk pyvdhftkmk |
| | venkklgnkp ltnqakliln |
| 541 | gaygkfgtkq nkeekdlimd knglltftgs vteyegkefy |
| | rpyasfvtay grlqlwnaii |
| 601 | yavgvenfly cdtdsiycnr evnsliedmn aigetidkti |
| | lgkwdvehvf dkfkvlgqkk |
| 661 | ymyhdckedk tdlkccglps darkiiigqg fdefylgknv |
| | egkkqrkkvi ggcllldtlf |
| 721 | tikkimf |

### Nanopores

Nanopores generated by both naturally-occurring, and non-naturally occurring (e.g., engineered or recombinant) pore-forming proteins find use herein. A wide range of pore-forming proteins are known in the art that can be used to generate nanopores useful for nanopore detection of the ion flow altering tags of the present disclosure. Biological nanopores of the include OmpG from E. coli, sp., Salmonella sp., Shigella sp., and Pseudomonas sp., and alpha hemolysin from S. aureus sp., MspA from M. smegmatis sp. Representative pore forming proteins include, but are not limited to, α-hemolysin, β-hemolysin, γ-hemolysin, aerolysin, cytolysin, leukocidin, melittin, MspA porin and porin A. The nanopores may be wild-type nanopores, variant nanopores, or modified variant nanopores.

Variant nanopores can be engineered to possess characteristics that are altered relative to those of the parental enzyme. See, for example, U.S. patent application Ser. No. 14/924,861 filed Oct. 28, 2015, entitled “alpha-Hemolysin Variants with Altered Characteristics,” and U.S. patent application Ser. No. 15/492,214 filed Apr. 20, 2017, entitled “alpha-Hemolysin variants and Uses Thereof”, which are incorporated herein by reference in its entirety.

Other variant nanopores are described, for example, in U.S. patent application Ser. No. 15/638,273, filed on Jun. 29, 2017, titled “Long Lifetime Alpha-Hemolysin Nanopores,” which is incorporated herein by reference in its entirety. In other example embodiments, the alpha-hemolysins of an alpha-hemolysin nanopore may be modified as described in International Patent Application No. PCT/EP2017/057433, filed on Mar. 29, 2017, titled “Nanopore Protein Conjugates and Uses Thereof,” which is incorporated herein by reference in its entirety.

The pore-forming protein, α-hemolysin from Staphylococcus aureus (also referred to herein as “α-HL”), is one of the most-studied members of the class of pore-forming proteins, and has been used extensively in creating nanopore devices. (See, e.g., U.S. Publication Nos. 2015/0119259, 2014/0134616, 2013/0264207, and 2013/0244340.) α-HL also has been sequenced, cloned, extensively characterized structurally and functionally using a wide range of techniques including site-directed mutagenesis and chemical labelling (see, e.g., Valeva et al. (2001), and references cited therein).

The amino acid sequence of the naturally occurring (i.e., wild type) α-HL pore forming protein subunit shown below.

| Wild-Type α-HL amino acid sequence | |
|---|---|
| (SEQ ID NO: 4) | |
| ADSDINIKTG TTDIGSNTTV KTGDLVTYDK ENGMHKKVFY SFIDDKNHNK KLLVIRTKGT | 60 |
| IAGQYRVYSE EGANKSGLAW PSAFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI FNNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWTDRSSE RYKIDWEKEE MTNGLSAWSH | 300 |
| PQFEK | 305 |

The above wild-type α-HL amino acid sequence is the mature sequence suitable for determining the location of substitutions and therefore does not include the initial methionine residue. In some embodiments, subunits of α-HL are truncated at amino acid G294, and optionally include a C-terminal SpyTag peptide fusion as disclosed below.

A variety of non-naturally occurring α-HL pore forming proteins have been made including, without limitation, variant α-HL subunits comprising one or more of the following substitutions: H35G, H144A, E111N, M113A, D127G, D128G, T129G, K131G, K147N, and V149K. Properties of these various engineered α-HL pore polypeptides are described in, e.g., U.S. Published Patent Application Nos. 2017/0088588, 2017/0088890, 2017/0306397, and 2018/0002750, each of which is hereby incorporated by reference herein.

### Attachment of Polymerase to Nanopore

It is well-known that a heptameric complex of α-HL monomers spontaneously forms a nanopore that embeds in and creates a pore through a lipid bilayer membrane. It has been shown that heptamers of α-HL comprising a ratio of 6:1 native α-HL to mutant α-HL can form nanopores (see, e.g., Valeva et al. (2001) “Membrane insertion of the heptameric staphylococcal alpha-toxin pore—A domino-like structural transition that is allosterically modulated by the target cell membrane,” J. Biol. Chem. 276(18): 14835-14841, and references cited therein). One α-HL monomer unit of the heptameric pore can be covalently conjugated with a DNA-polymerase using a SpyCatcher/SpyTag conjugation method as described in WO 2015/148402, which is hereby incorporated by reference herein (see also, Zakeri and Howarth (2010), J. Am. Chem. Soc. 132:4526-7). Briefly, a SpyTag peptide is attached as a recombinant fusion to the C-terminus of the 1× subunit of α-HL, and a SpyCatcher protein fragment is attached as a recombinant fusion to the N-terminus of the strand-extending enzyme, e.g., Pol6 DNA polymerase. The SpyTag peptide and the SpyCatcher protein fragment undergo a reaction between a lysine residue of the SpyCatcher protein and an aspartic acid residue of the SpyTag peptide that results in a covalent linkage conjugating the two the α-HL subunit to the enzyme.

Generally, the α-HL subunits are used to prepare heptameric α-HL nanopores with the same methods used with wild-type or other engineered α-HL proteins known in the art. Accordingly, in some embodiments, the compounds of the present disclosure can be used with a nanopore device. The heptameric α-HL nanopore has six subunits, each having no linker for attaching a polymerase, and one subunit, which has a C-terminal fusion (beginning at position 294 of the truncated wild-type sequence) that includes the SpyTag peptide, AHIVMVDAYK (SEQ ID NO: 5). The SpyTag peptide allows conjugation of the nanopore to a SpyCatcher-modified strand-extending enzyme, such as a Pol6 DNA polymerase.

In some embodiments, the C-terminal SpyTag peptide fusion of the mutants comprises a linker peptide (e.g., GSSGGSSGG (SEQ ID NO: 6)), a SpyTag peptide (e.g., AHIVMVDAYKPTK (SEQ ID NO: 7)), and a terminal His tag (e.g., KGHHHHHH (SEQ ID NO: 8)). Thus, the C-terminal SpyTag peptide fusion that comprises the amino acid sequence: GSSGGSSGGAHIVMVDAYKPTKKGHHHHHH (SEQ ID NO: 9). In some embodiments, the C-terminal SpyTag peptide fusion is attached at position 294 of one subunit which is truncated relative to the wild-type α-HL subunit sequence. (See, e.g., C-terminal SpyTag peptide fusion of SEQ ID NO: 2 as disclosed in WO2017125565A1, which is hereby incorporated by reference herein.).

An exemplary method for attaching a polymerase to a nanopore involves attaching a linker molecule to a nanopore or mutating a nanopore to have an attachment site and then attaching a polymerase to the attachment site or attachment linker. The polymerase is attached to the attachment site or attachment linker before the nanopore is inserted in the membrane. In some cases, a Conjugate is inserted into a lipid membrane disposed over wells and/or electrodes of a biochip.

In some instances, the polymerase is expressed as a fusion protein that comprises a SpyCatcher polypeptide, which can be covalently bound to a nanopore that comprises a SpyTag peptide (Zakeri et al. PNAS109:E690-E697 [2012]).

The Conjugate, e.g., polymerase-nanopore complex, can be formed in any suitable way. Attaching polymerases to nanopores may be achieved using the SpyTag/SpyCatcher peptide system (Zakeri et al. PNAS109:E690-E697 [2012]) native chemical ligation (Thapa et al., Molecules 19:14461-14483 [2014]), sortase system (Wu and Guo, J Carbohydr Chem 31:48-66 [2012]; Heck et al., Appl Microbiol Biotechnol 97:461-475 [2013]), transglutaminase systems (Dennler et al., Bioconjug Chem 25:569-578 [2014]), formylglycine linkage (Rashidian et al., Bioconjug Chem 24:1277-1294 [2013]), or other chemical ligation techniques known in the art.

In some instances, the polymerase is linked to the nanopore using Solulink™ chemistry. Solulink™ can be a reaction between HyNic (6-hydrazino-nicotinic acid, an aromatic hydrazine) and 4FB (4-formylbenzoate, an aromatic aldehyde). In some instances, the polymerase is linked to the nanopore using Click chemistry (available from LifeTechnologies, for example).

In some cases, zinc finger mutations are introduced into the nanopore molecule and then a molecule is used (e.g., a DNA intermediate molecule) to link the Pol6 polymerase to the zinc finger sites on the nanopore, e.g., α-hemolysin.

Additionally, a polymerase can be attached to a nanopore, e.g., aHL, OmpG, by means of a linker molecule that is attached to a nanopore at an attachment site. In some cases, the polymerase is attached to the nanopore with molecular staples. In some instances, molecular staples comprise three amino acid sequences (denoted linkers A, B and C). Linker A can extend from a nanopore monomer, Linker B can extend from the polymerase, and Linker C then can bind Linkers A and B (e.g., by wrapping around both Linkers A and B) and thus linking the polymerase to the nanopore. Linker C can also be constructed to be part of Linker A or Linker B, thus reducing the number of linker molecules.

Other linkers that may find use in attaching the polymerase to a nanopore are direct genetic linkage (e.g., (GGGGS)₁₋₃ amino acid linker (SEQ ID NO: 1)), transglutaminase mediated linking (e.g., RSKLG (SEQ ID NO: 2)), sortase mediated linking, and chemical linking through cysteine modifications. Specific linkers contemplated as useful herein are (GGGGS)₁₋₃ (SEQ ID NO: 1), K-tag (RSKLG (SEQ ID NO: 2)) on N-terminus, ATEV site (12-25), ATEV site+N-terminus of SpyCatcher (12-49).

Alternatively, an α-HL monomer can be engineered with cysteine residue substitutions inserted at numerous positions allowing for covalent modification of the protein through maleimide linker chemistry (see, e.g., Valeva et al. (2001)). For example, the single α-HL subunit can be modified with a K46C mutation which then is easily modified with a linker allowing the use of tetrazine-trans-cyclooctene click chemistry to covalently attach a Bst2.0 variant of DNA polymerase to the heptameric 6:1 nanopore. Such an embodiment is described in U.S. application Ser. No. 15/439,173, filed Feb. 22, 2017, entitled “Pore-forming Protein Conjugate Compositions and Methods,” which is hereby incorporated by reference herein.

Other methods for attaching strand-extending enzymes to nanopores include native chemical ligation (Thapa et al., Molecules 19:14461-14483 [2014]), sortase system (Wu and Guo, J Carbohydr Chem 31:48-66 [2012]; Heck et al., Appl Microbiol Biotechnol 97:461-475 [2013]), transglutaminase systems (Dennler et al., Bioconjug Chem 25:569-578 [2014]), formylglycine linkage (Rashidian et al., Bioconjug Chem 24:1277-1294 [2013]), or other chemical ligation techniques known in the art. Polymerases may also be attached to nanopores using methods described, for example, in PCT/EP2017/057002 (published as WO2017/162828; Genia Technologies, Inc. and F. Hoffmann-La Roche AG), PCT/US2013/068967 (published as WO2014/074727; Genia Technologies, Inc.), PCT/US2005/009702 (published as WO2006/028508; President and Fellows of Harvard College), and PCT/US2011/065640 (published as WO2012/083249; Columbia University).

Nanopore sequencing with the aid of a polymerase is accomplished by sequencing complexes, which are formed by the association of a Primed Template (e.g., a target polynucleotide associated with purification moiety) to a Conjugate (i.e., a polymerase-nanopre complex). In some embodiments, the polymerase-pore complex is subsequently linked to a template to form the sequencing complex, which is subsequently inserted into a lipid bilayer.

Measurements of ionic current flow through a nanopore are made across a nanopore that has been inserted (e.g., by electroporation) into a lipid membrane. The nanopore can be inserted by a stimulus signal such as electrical stimulus, pressure stimulus, liquid flow stimulus, gas bubble stimulus, sonication, sound, vibration, or any combination thereof. In some cases, the membrane is formed with aid of a bubble and the nanopore is inserted in the membrane with aid of an electrical stimulus. In other embodiments, the nanopore inserts itself into the membrane. Methods for assembling a lipid bilayer, and sequencing nucleic acid molecules can be found in PCT Patent Publication Nos. WO2011/097028 and WO2015/061510, which are incorporated herein by reference in their entirety.

In some example embodiments, the nanopore characteristics are altered relative to the wild-type nanopore. In some embodiments, the variant nanopore of the nanopore sequencing complex is engineered to reduce the ionic current noise of the parental nanopore from which it is derived. An example of a variant nanopore having an altered characteristic is the OmpG nanopore having one or more mutations at the constriction site (International Patent Application No. PCT/EP2016/072224, entitled “OmpG Variants”, filed on Sep. 20, 2016, which is incorporated by reference herein in its entirety), which decrease the ionic noise level relative to that of the parent OmpG. The reduced ionic current noise provides for the use of these OmpG nanopore variants in single molecule sensing of polynucleotides and proteins. In other embodiments, the variant OmpG polypeptide can be further mutated to bind molecular adapters, which while resident in the pore slow the movement of analytes, e.g., nucleotide bases, through the pore and consequently improve the accuracy of the identification of the analyte (Astier et al., J Am Chem Soc 10.1021/ja057123+, published online on Dec. 30, 2005).

Modified variant nanopores are typically multimeric nanopores whose subunits have been engineered to affect inter-subunit interaction (U.S. patent application Ser. No. 15/274,770, entitled “Alpha-Hemolysin Variants”, filed on Sep. 23, 2016, which is incorporated by reference herein in its entirety). Altered subunit interactions can be exploited to specify the sequence and order with which monomers oligomerize to form the multimeric nanopore in a lipid bilayer. This technique provides control of the stoichiometry of the subunits that form the nanopore. An example of a multimeric nanopore whose subunits can be modified to determine the sequence of interaction of subunits during oligomerization is an aHL nanopore.

In some example embodiments, a single polymerase is attached to each nanopore. In other embodiments, two or more polymerases are attached to a monomeric nanopore or to a subunit of an oligomeric nanopore.

### Nanopore Devices

Nanopore devices and methods for making and using them in nanopore detection applications, such as nanopore sequencing using ion flow altering tagged nucleotides are known in the art (See, e.g., U.S. Pat. Nos. 7,005,264 B2; 7,846,738; 6,617,113; 6,746,594; 6,673,615; 6,627,067; 6,464,842; 6,362,002; 6,267,872; 6,015,714; 5,795,782; and U.S. Publication Nos. 2015/0119259, 2014/0134616, 2013/0264207, 2013/0244340, 2004/0121525, and 2003/0104428, each of which are hereby incorporated by reference in their entirety). Generally, the nanopore devices comprise a poreforming protein embedded in a lipid-bilayer membrane, wherein the membrane is immobilized or attached to a solid substrate which comprises a well or reservoir. The pore of the nanopore extends through the membrane creating a fluidic connection between the cis and trans sides of the membrane. Typically, the solid substrate comprises a material selected from the group consisting of polymer, glass, silicon, and a combination thereof. Additionally, the solid substrate comprises adjacent to the nanopore, a sensor, a sensing circuit, or an electrode coupled to a sensing circuit, optionally, a complementary metal-oxide semiconductor (CMOS), or field effect transistor (FET) circuit. Typically, there are electrodes on the cis and trans sides of the membrane that allow for a DC or AC voltage potential to be set across the membrane which generates a baseline current flow (or O.C. current level) through the pore of the nanopore. The presence of a tag, such as described in U.S. Provisional Application 62/636,807, filed 28 Feb. 2018, titled “Tagged Nucleoside Compounds Useful For Nanopore Detection”, International Patent Application PCT/EP2016/070198, filed 26 Aug. 2016 titled “Polypeptide Tagged Nucleotides and Uses Thereof”, US Patent Application Publications US 2013/0244340 A1, published Sep. 19, 2013 titled “Nanopore Based Molecular Detection and Sequencing”, US 2013/0264207 A1, published Oct. 10, 2013 titled “DNA Sequencing By Synthesis Using Modified Nucleotides And Nanopore Detection”, and US 2014/0134616 A1, published May 14, 2014 titled “Nucleic Acid Sequencing Using Tags”, results in change in positive ion flow through the nanopore and thereby generates a measurable change in current level across the electrodes relative to the O.C. current of the nanopore.

It is contemplated that the ion flow altering tag compounds (i.e., tagged nucleotides) can be used with a wide range nanopore devices comprising nanopores generated by both naturally-occurring, and non-naturally occurring (e.g., engineered or recombinant) pore-forming proteins. A wide range of pore-forming proteins are known in the art that can be used to generate nanopores useful for nanopore detection of the ion flow altering tags of the present disclosure. Representative pore forming proteins include, but are not limited to, α-hemolysin, β-hemolysin, γ-hemolysin, aerolysin, cytolysin, leukocidin, melittin, MspA porin and porin A.

The amino acid sequence of the naturally occurring (i.e., wild type) α-HL pore forming protein subunit shown below.

| Wild-Type α-HL amino acid sequence | |
|---|---|
| (SEQ ID NO: 4) | |
| ADSDINIKTG TTDIGSNTTV KTGDLVTYDK ENGMHKKVFY SFIDDKNHNK KLLVIRTKGT | 60 |
| IAGQYRVYSE EGANKSGLAW PSAFKVQLQL PDNEVAQISD YYPRNSIDTK EYMSTLTYGF | 120 |
| NGNVTGDDTG KIGGLIGANV SIGHTLKYVQ PDFKTILESP TDKKVGWKVI FNNMVNQNWG | 180 |
| PYDRDSWNPV YGNQLFMKTR NGSMKAADNF LDPNKASSLL SSGFSPDFAT VITMDRKASK | 240 |
| QQTNIDVIYE RVRDDYQLHW TSTNWKGTNT KDKWTDRSSE RYKIDWEKEE MTNGLSAWSH | 300 |
| PQFEK | |

The above wild-type α-HL amino acid sequence is the mature sequence suitable for determining the location of substitutions described herein and therefore does not include the initial methionine residue. In some embodiments, the mutant subunits of α-HL in addition to including the mutations disclosed herein are also truncated at amino acid G294, and optionally include a C-terminal SpyTag peptide fusion as disclosed below.

A variety of non-naturally occurring α-HL pore forming proteins have been made including, without limitation, variant α-HL subunits comprising one or more of the following substitutions: H35G, H144A, E111N, M113A, D127G, D128G, T129G, K131G, K147N, and V149K. Properties of these various engineered α-HL pore polypeptides are described in, e.g., U.S. Published Patent Application Nos. 2017/0088588, 2017/0088890, 2017/0306397, and 2018/0002750, each of which is hereby incorporated by reference herein.

### Methods for Sequencing Polynucleotides

As described elsewhere herein, the molecules being characterized using the variant Pol6 polymerases of the Pol6 nanopore sequencing complexes described herein can be of various types, including charged or polar molecules such as charged or polar polymeric molecules. Specific examples include ribonucleic acid (RNA) and deoxyribonucleic acid (DNA) molecules. The DNA can be a single-strand DNA (ssDNA) or a double-strand DNA (dsDNA) molecule. Ribonucleic acid can be reversed transcribed then sequenced.

In certain example embodiments, provided are methods for sequencing nucleic acids at high concentrations of salt using the polymerase-template complexes prepared according to the methods provided herein, i.e., at high concentrations of salt and in the absence of nucleotides. The polymerase-template complexes are subsequently attached to a nanopore to form a nanopore sequencing complex, which detects polynucleotide sequences. In other example embodiments, provided are methods for sequencing nucleic acids using the polymerase-template complexes prepared according to the methods provided herein, such as forming the polymerase-template complexes using low nucleotide concentrations, at high temperatures, and in the presence of excess polymerase. The polymerase-template complexes are subsequently attached to a nanopore to form a nanopore sequencing complex, which detects polynucleotide sequences.

The nanopore sequencing complexes comprising polymerase-template complexes prepared according to the compositions and methods provided herein, can be used for determining the sequence of nucleic acids at high concentrations of salt using other nanopore sequencing platforms known in the art that utilize enzymes in the sequencing of polynucleotides. Likewise, the nanopore sequencing complexes comprising polymerase-template complexes prepared according to the compositions and methods provided, can be used for determining the sequence of nucleic acids at, for example, high temperatures using other nanopore sequencing platforms known in the art that utilize enzymes in the sequencing of polynucleotides. For example, nanopore sequencing complexes comprising the polymerase-template complexes prepared according to the methods described herein can be used for sequencing nucleic acids according to the helicase and exonuclease-based methods of Oxford Nanopore (Oxford, UK), Illumina (San Diego, Calif.), and the nanopore sequencing-by-expansion of Stratos Genomics (Seattle, Wash.).

In some example embodiments, sequencing of nucleic acids comprises preparing nanopore sequencing complexes comprising polymerase-template complexes prepared according to the methods described herein, and determining polynucleotide sequences at high concentrations of salt using tagged nucleotides as is described in PCT/US2013/068967 (entitled “Nucleic Acid Sequencing Using Tags” filed on Nov. 7, 2013, which is herein incorporated by reference in its entirety). For example, a nanopore sequencing complex that is situated in a membrane (e.g., a lipid bilayer) adjacent to or in sensing proximity to one or more sensing electrodes, can detect the incorporation of a tagged nucleotide by a polymerase at a high concentration of salt as the nucleotide base is incorporated into a strand that is complementary to that of the polynucleotide associated with the polymerase, and the tag of the nucleotide is detected by the nanopore. The polymerase-template complex can be associated with the nanopore as provided herein.

Tags of the tagged nucleotides can include chemical groups or molecules that are capable of being detected by a nanopore. Examples of tags used to provide tagged nucleotides are described at least at paragraphs [0414] to [0452] of PCT/US2013/068967. Nucleotides may be incorporated from a mixture of different nucleotides, e.g., a mixture of tagged dNTPs where N is adenosine (A), cytidine (C), thymidine (T), guanosine (G) or uracil (U). Alternatively, nucleotides can be incorporated from alternating solutions of individual tagged dNTPs, i.e., tagged dATP followed by tagged dCTP, followed by tagged dGTP, etc. Determination of a polynucleotide sequence can occur as the nanopore detects the tags as they flow through or are adjacent to the nanopore, as the tags reside in the nanopore and/or as the tags are presented to the nanopore. The tag of each tagged nucleotide can be coupled to the nucleotide base at any position including, but not limited to a phosphate (e.g., gamma phosphate), sugar or nitrogenous base moiety of the nucleotide. In some cases, tags are detected while tags are associated with a polymerase during the incorporation of nucleotide tags. The tag may continue to be detected until the tag translocates through the nanopore after nucleotide incorporation and subsequent cleavage and/or release of the tag. In some cases, nucleotide incorporation events release tags from the tagged nucleotides, and the tags pass through a nanopore and are detected. The tag can be released by the polymerase, or cleaved/released in any suitable manner including without limitation cleavage by an enzyme located near the polymerase. In this way, the incorporated base may be identified (i.e., A, C, G, T or U) because a unique tag is released from each type of nucleotide (i.e., adenine, cytosine, guanine, thymine or uracil). In some situations, nucleotide incorporation events do not release tags. In such a case, a tag coupled to an incorporated nucleotide is detected with the aid of a nanopore. In some examples, the tag can move through or in proximity to the nanopore and be detected with the aid of the nanopore.

Thus, in one aspect, a method is provided for sequencing a polynucleotide from a sample, e.g. a biological sample, with the aid of a nanopore sequencing complex at a high concentration of salt. The sample polynucleotide is combined with the polymerase in a solution comprising a high concentration of salt and being essentially free of nucleotides to provide the polymerase-template complex portion of the nanopore sequencing complex. In one embodiment, the sample polynucleotide is a sample ssDNA strand, which is combined with a DNA polymerase to provide a polymerase-DNA complex, e.g., a Po16-DNA complex.

In some embodiments, nanopore sequencing of a polynucleotide sample is performed by providing a polymerase-template complex, e.g., Po16-template or variant Po16-template complex in a solution comprising a high concentration of salt, e.g., greater than 100 mM, and being essentially free of nucleotides; attaching the polymerase-template complex to a nanopore to form a nanopore-sequencing complex; and providing nucleotides to initiate template-dependent strand synthesis. The nanopore portion of the sequencing complex is positioned in the membrane adjacent to or in proximity of a sensing electrode, as described elsewhere herein. The resulting nanopore sequencing complex is capable of determining the sequence of nucleotide bases of the sample DNA at a high concentration of salt as described elsewhere herein. In other embodiments, the nanopore sequencing complex determines the sequence of double stranded DNA. In other embodiments, the nanopore sequencing complex determines the sequence of single stranded DNA. In yet other embodiments, nanopore sequencing complex determines the sequence of RNA by sequencing the reverse transcribed product.

In some embodiments, a method is provided for nanopore sequencing. The method comprises (a) providing a polymerase-template complex in a solution comprising a high concentration of salt, e.g., at least 100 mM, and being free of nucleotides; (b) combining the polymerase-template complex with a nanopore to form a nanopore-sequencing complex; (c) providing tagged nucleotides to the nanopore sequencing complex to initiate template-dependent nanopore sequencing; and (d) detecting with the aid of the nanopore, a tag associated with each of the tagged nucleotides during incorporation of each of the nucleotides to determine that sequence of the template. The polymerase of the polymerase-template complex can be a wild-type or a variant polymerase that retains polymerase activity at high concentration of salt. Examples of polymerases that find use in the compositions and methods described herein include the salt-tolerant polymerases described elsewhere herein. In some embodiments, the polymerase of the polymerase-template complex is a Pol6 polymerase that has an amino acid sequence that is at least 70% identical to SEQ ID NO: 3.

In some embodiments, a method for nanopore sequencing a nucleic acid sample is provided. The method comprises using nanopore sequencing complexes comprising the variant Pol6 polymerases provided herein. In one embodiment, the method comprises providing tagged nucleotides to a Pol6 nanopore sequencing complex, and carrying out a polymerization reaction to incorporate the nucleotides in a template-dependent manner, and detecting the tag of each of the incorporated nucleotides to determine the sequence of the template DNA.

In one embodiment, tagged nucleotides are provided to a Pol6 nanopore sequencing complex comprising a variant Pol6 polymerase provided herein, and carrying out a polymerization reaction with the aid of the variant Pol6 enzyme of said nanopore sequencing complex, to incorporate tagged nucleotides into a growing strand complementary to a single stranded nucleic acid molecule from the nucleic acid sample; and detecting, with the aid of nanopore, a tag associated with said individual tagged nucleotide during incorporation of the individual tagged nucleotide, wherein the tag is detected with the aid of said nanopore while the nucleotide is associated with the variant Pol6 polymerase.

In one aspect, a method is provided for sequencing a polynucleotide from a sample, e.g., a biological sample, with the aid of a nanopore sequencing complex at a high temperature and at a low concentration of nucleotides. For example, the sample polynucleotide is combined with the polymerase in a solution having a high temperature and having a low concentration of nucleotides. In one embodiment, the sample polynucleotide is a sample ssDNA strand, which is combined with a DNA polymerase to provide a polymerase-DNA complex, e.g., a Po16-DNA complex. The temperature may be above room temperature, such as at about 40° C., as described herein. The nucleotide concentration, for example, may be about 1.2 μM, as described herein. Further, the solution may include a high concentration of the polymerase, such as being saturated with the polymerase. The polymerase can be a variant polymerase as described herein.

In certain example aspects, a method is provided for nanopore-based sequencing of a polynucleotide template. The method includes forming a polymerase-template complex, as described herein, in a solution including a low concentration of nucleotides, the solution having a high temperature, such as above room temperature. For example, the temperature may be about 40° C., as described herein. The method includes combining the formed polymerase-template complex with a nanopore to form a nanopore-sequencing complex. Tagged nucleotides can then be provided to the nanopore sequencing complex to initiate template-dependent nanopore sequencing of the template at the high temperature. With the aid of the nanopore, a tag associated with each of the tagged nucleotides during incorporation of each of the tagged nucleotides while each of the tagged nucleotides is associated with the polymerase is detected, thereby determining the sequence of the polynucleotide template. In certain examples, forming the polymerase-template complex includes saturating the solution with the polymerase of the polymerase-template complex. The nucleotide concentration can be 0.8 μM to 2.2 μM, such as about 1.2 μM. The temperature, for example, can be about 35° C. to 45° C., such as about 40° C.

Other embodiments of the sequencing method that comprise the use of tagged nucleotides with the present nanopore sequencing complexes for sequencing polynucleotides are provided in WO2014/074727, which is incorporated herein by reference in its entirety.

Sequencing nucleic acids using AC waveforms and tagged nucleotides is described in US Patent Publication US2014/0134616 entitled “Nucleic Acid Sequencing Using Tags”, filed on Nov. 6, 2013, which is herein incorporated by reference in its entirety. In addition to the tagged nucleotides described in US2014/0134616, sequencing can be performed using nucleotide analogs that lack a sugar or acyclic moiety, e.g., (S)-Glycerol nucleoside triphosphates (gNTPs) of the five common nucleobases: adenine, cytosine, guanine, uracil, and thymidine (Horhota et al. Organic Letters, 8:5345-5347 [2006]).

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds).

### EXAMPLES

### Example 1—Annealed Template Preparation

This Example relates to a method for the preparation of an Annealed Template.

In an Eppendorf tube (1.5 mL) the Primer mix was prepared by mixing 25.0 μL, Anneal Buffer (500 mM NaCl, 100 mM Tris, pH 8.0), 5.0 μL Enrichment Primer (20 μM), and 20 μL Nuclease-free water. The Enrichment Primer comprises a nucleotide sequence that is complementary to a portion of a template DNA, at least one uracil residue, and a nucleotide sequence linked to a purification moiety.

In a separate tube for each sample to be sequenced, 10 μL of Primer mix was added to 40 μL of sample DNA (25 nM) and mixed.

Each tube was placed in a thermocycler and incubated using the following protocol: 30 seconds at 45° C., Cool to 20° C. to 4° C. at a rate of −0.1° C./sec. The temperature was held at 4° C. until the tubes were removed from the thermocycler and placed on ice.

To each 50 μL of anneal reaction, 75 μL of AMPure XP beads (Beckman Coulter) were added (DNA: bead ratio=1:1.5). The tube was vortexed for 5 seconds, then briefly spun. The reaction was allowed to incubate at room temperature for 10 minutes, after which the tube was placed on a magnetic rack at room temperature for several minutes until the supernatant cleared. While in this example this clean-up step is included, it is optional and may be omitted. The supernatant was carefully removed, and 200 μL, 80% ethanol was added. The tubes were placed back on the magnetic rack and the ethanol was removed after several minutes. The beads were carefully washed one more time with ethanol and the ethanol removed as above. The beads were resuspended in 10 μL buffer (75 mM KGlu, 20 mM HEPES pH 7.5, 0.01% (w/v) Tween-20, 5 mM TCEP, 8% (w/v) Trehalose, and 10 μM blocked Cytosine (e.g., dCpCpp (dCMPCPP) 2′-Deoxycytidine-5′-[(α,β)-methyleno]triphosphate, Sodium salt. dpCpCpp is an α,β non-hydrolysable analog of dCTP), the tube briefly vortexed and spun to bring the contents to the bottom of the tube. The tube is incubated at room temperature for 5 minutes. The tubes were then placed on the magnetic rack and magnetized for 1 min until supernatant is clear. The supernatant is carefully removed and transferred to a fresh 0.2 mL PCR tube. The supernatant contains the Annealed Template.

### Example 2—Eintopf Preparation and Complex Formation

This example relates to a method for the preparation of an Eintopf composition. Eintopf is a solution of the Annealed Template, and Conjugate that allows for the formation of the Sequencing Complex.

A Conjugate was prepared as described herein using the SpyTag/SpyCatcher system. Ten microliters of a 0.4 μM Conjugate in Eintopf Buffer (75 mM KGlu, 20 mM HEPES pH 7.5, 0.01% (w/v) Tween-20, 5 mM TCEP, 8% (w/v) Trehalose, and 10 μM blocked Cytosine) was added to each Annealed Template as prepared in Example 1. The resulting conjugate:template ratio was approximately 4:1, with the final Conjugate concentration being 400 nM in a total volume of 20 μL (i.e., 10 μL of Annealed Template and 10 μL of the Conjugate).

The tubes were then placed in a thermocycler and incubated for 30 minutes at 36° C., then chilled to 4° C. This allows for the spontaneous isopeptide bond to form between the SpyTag and the SpyCatcher moieties. Upon completion, tubes were removed from the thermocycler and placed on ice or held at 4° C. If the Sequencing Complexes were not to be used the day of preparation of the Eintopf composition, they were stored at −80° C. until ready for Enrichment as described in Example 3, below.

### Example 3—Complex Enrichment

This example relates to the enrichment of the Sequencing Complexes from the Eintopf as prepared in Example 2, above.

### Pre-Enrichment—Bead Washing

Into a fresh 1.5 mL tube, 50 μL, of KilobaseBINDER Enrichment Beads (ThermoFisher) were aliquoted for each sample to be sequenced. The tubes were placed on a magnetic rack, and the beads allowed to separate for 2-3 minutes until supernatant was clear. The supernatant was removed, being careful not to disturb the bead bed. While tubes were still on the magnetic rack, 500 μL of Eintopf Buffer was added. The supernatant was slowly remove, again being careful not to disturb the bead bed. Another 500 μL of Eintopf Buffer was added and removed, being careful not to disturb the bead bed or let the beads dry out. The tubes were then removed from the magnetic rack and spun briefly to bring contents to the bottom of the tube. The tube was again placed on the magnetic rack and magnetized for 1 minute until the supernatant was clear. Any residual supernatant was carefully removed, 20 μL of Eintopf Buffer was added, and the tubes removed from the magnetic rack. The tubes were vortexed vigorously to resuspend the beads, and spun briefly in a bench top microcentrifuge to bring contents to the bottom of the tube. The beads are now washed and ready for use in the Enrichment protocol.

### Enrichment

Preheat the thermomixer to 20° C. To the washed beads, 20 μL of Eintopf composition prepared in Example 2 was added and mixed thoroughly. The tubes were placed in a programmable thermomixer and incubated for 10 min at 20° C. at 1,200 rpm. The tubes are removed and place on a magnetic rack and the beads allowed to separate for 2-3 minutes until the supernatant was clear. The supernatant was slowly removed, being careful not to disturb the bead bed. While tubes were still on the magnetic rack, add 500 μL Wash Buffer (300 mM KGlu, 20 mM HEPES pH 7.5, 0.01% (w/v) Tween-20, 5 mM TCEP, 8% (w/v) Trehalose, and 10 μM blocked C). The supernatant was slowly removed, again being careful not to disturb the bead bed. The tubes were removed from the magnetic rack, and 79 μL, Wash Buffer and 1 μL USER enzyme were added to the beads. The tubes were thoroughly mixed and spun briefly to bring the contents to the bottom of the tube. Tubes were placed in the thermomixer and incubated 10 min at 20° C. at 1,200 rpm. The tubes are removed, briefly spun, and place on a magnetic rack and the beads allowed to separate for 2-3 minutes until the supernatant was clear. The supernatant was slowly removed, again being careful not to disturb the bead bed, and transferred to a fresh 1.5 mL tube. Two μL of recovered DNA was used to quantify the dsDNA associated with the Sequencing Complexes using the Qubit High Sensitivity (HS) dsDNA assay (ThermoFisher), according to the manufacturer's recommended protocol. The concentration was converted to nM using the following equation:

Qubit conc (ng/μL)×1e6/[average fragment length (nt)×660 (g/mol)]=conc (nM)

The concentration of the DNA was then adjusted to 6 nM by adding an appropriate amount of Wash Buffer. The 6 nM sample was then stored on ice or held at 4° C. if it is to be used within 12 hours, or stored at −80° C. until ready for use in sequencing.

The composition at this point is referred to as the Enriched Sequencing Complex, which is the primed template (e.g., Annealed Template, self-priming template, etc.), bound to the Conjugate.

### Example 4—Preparation for Sequencing

This example describes the preparation of the Enriched Sequencing Complex for use in nanopore sequencing.

Examples of dilution volumes and loading concentrations are shown in the table below:

TABLE

| Loading concentration: | 0.2 nM | 0.4 nM | 0.6 nM | 1.0 nM | 2.0 nM |
|---|---|---|---|---|---|
| Enriched Sequencing | 10 μL | 20 μL | 30 μL | 50 μL | 100 μL |
| Complex | | | | | |
| Dilution Buffer* | 290 μL | 280 μL | 270 μL | 250 μL | 200 μL |
| TOTAL | 300 μL | 300 μL | 300 μL | 300 μL | 300 μL |

Dilution Buffer: 20 mM HEPES, 300 mM KGlu, 0.001% Tween 20, 8% Trehalose, 5 mM TCEP, 10 μM C—BN (blocked nucleotide), 10 mM MgCl2, 15 mM LiAce, 0.5 mM EDTA, 0.05% Proclin300, pH 7.5

The samples were stored at 4° C. or on ice until ready to proceed to sequencing within 12 hours of preparation.

### Example 5—Sequencing

This example describes the use of the Enriched Sequencing Complex in nanopore sequencing.

A biochip is provided comprising a plurality of wells wherein a bilayer has been disposed over the plurality of wells. The bilayers were formed as described in PCT/US14/61853 filed 23 Oct. 2014. The nanopore device (or sensor) used to detect a molecule (and/or sequence a nucleic acid) was set-up as described in WO2013123450.

The electrodes were conditioned and phospholipid bilayers were established on the chip as described in PCT/US2013/026514. The diluted Enriched Sequencing Complex provided for in Example 4, above, was flowed over a biochip and Sequencing Complexes were inserted as described in PCT/US14/61853 filed 23 Oct. 2014, or PCT/US2013/026514 (published as WO2013/123450). Although these protocols describe the use of a pore-polymerase complex without the Annealed Template bound, the same protocols can be used for a Sequencing Complex.

Nanopore ion flow measurements: After insertion of the complex into the membrane, the solution on the cis side is replaced by an osmolarity buffer: 10 mM MgCl₂, 15 mM LiOAc, 5 mM TCEP, 0.5 mM EDTA, 20 mM HEPES, 300 mM potassium glutamate, pH 7.8, 20° C. 500 μM of each of the set of the 4 different nucleotide substrates is added. The trans side buffer solution is: 10 mM MgCl₂, 15 mM LiOAc, 0.5 mM EDTA, 20 mM HEPES, 380 mM potassium glutamate, pH 7.5, 20° C. These buffer solutions are used as the electrolyte solutions for the nanopore ion flow measurements. A Pt/Ag/AgCl electrode setup is used and an AC current of a 210 mV pk-to-pk waveform applied at 976 Hz. AC current has certain advantages for nanopore detection as it allows for the tag to be repeatedly directed into and then expelled from the nanopore thereby providing more opportunities to measure signals resulting from the ion flow through the nanopore. Also, the ion flow during the positive and negative AC current cycles counteract each other to reduce the net rate of ion depletion from the cis side, and possible detrimental effects on signals resulting from this depletion.

The tag current level signal representing the distinct altered ion-flow event resulting from each different polymer moiety tag is observed as the tagged nucleotide is captured by the α-HL-Pol6 nanopore-polymerase conjugates primed with the DNA template. Plots of these events are recorded over time and analyzed. Generally, events that last longer than 10 ms indicate productive tag capture coincident with polymerase incorporation of the correct base complementary to the template strand.

All publications, patents, patent applications and other documents cited in this application are hereby incorporated by reference in their entireties for all purposes to the same extent as if each individual publication, patent, patent application or other document were individually indicated to be incorporated by reference for all purposes.

1. A method for isolating enriched Sequencing complexes, said method comprising:
(a) annealing an Enrichment Primer comprising a linker and a purification moiety to a sample DNA to form an Annealed Template Oligonucleotide;
(b) purifying the Annealed Template Oligonucleotide;
(c) combining a nanopore covalently linked to a polymerase with the Annealed Template Oligonucleotide to form a solution of the Annealed Template, and the nanopore covalently linked to the polymerase that allows for the formation of the Sequencing Complex;
(d) combining the solution from step (c) with a solid support capable of binding to the Purification Moiety; and
(e) cleaving the linker of the enrichment primer with an enzyme composition to release the Purification Moiety thereby releasing the Sequencing Complex to provide an Enriched Sequencing Complex solution.

2. A method for isolating enriched Sequencing complexes, said method comprising:
(a) combining a nanopore covalently linked to a polymerase with an Annealed Template Oligonucleotide comprising a linker and a Purification Moiety and allowing the nanopore covalently linked to the polymerase to bind the Annealed Template Oligonucleotide to form a Sequencing Complex;
(b) combining the Sequencing Complex with a solid support capable of binding to the Purification Moiety of the Annealed Template Oligonucleotide,
(c) separating the unbound Sequencing Complex Components from the bound Sequencing Complexes;
(d) cleaving the linker with an enzyme composition to release the Purification Moiety, wherein the purification moiety remains associated with the solid support, thereby releasing the Sequencing Complex, and
(e) separating the solid support from the Sequencing Complex to provide an Enriched Sequencing Complex solution.

3. (canceled)

4. The method of claim 1, wherein the linker comprises an abasic site or at least one uracil residue.

5. The method of claim 1, wherein the sample DNA is either linear, circular or self-priming.

6. The method of claim 5, wherein the sample DNA has been ligated to at least one adaptor.

7. The method of claim 5, wherein an adaptor has been ligated to each end of the sample DNA.

8. The method of claim 7, wherein the adaptors are dumbbell adaptors.

9. The method of claim 6, wherein the adaptor comprises a primer recognition sequence capable of binding to the Enrichment Primer.

10. The method of claim 1, wherein purifying the Annealed Template Oligonucleotide comprises binding to a solid support that selectively binds double stranded DNA.

11. The method of claim 10, wherein the sample DNA comprises a barcode.

12. The method of claim 1, wherein the solid support capable of binding to the Purification Moiety is a bead.

13. The method of claim 1, wherein the enzyme composition comprises an Endonuclease VIII, an Endonuclease III, a lyase, a glycolyase, or combinations thereof.

14. A method for preparing a biochip, said method comprising:
(a) isolating a Sequencing Complex according to; claim 1, and
(b) flowing the enriched Sequencing Complex over a lipid bilayer of said biochip; and
(c) applying a voltage to said chip sufficient to insert the Sequencing Complex in the lipid bilayer.

15. The method of claim 14, wherein said biochip has a density of said nanopore sequencing complexes of at least 500,000 nanopore sequencing complexes per 1 mm².
